(19) United States
(12) Patent Application Publication
(10) Pub. No.: US 2009/0054758 A1
(43) Pub. Date: Feb. 26, 2009

(54) Apparatus and Method for Reducing Interference

(76) Inventor: William James Ross Dunseath, Charlottesville, VA (US)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/885,533

(22) PCT Filed: Feb. 9, 2006
(86) PCT No.: PCT/EP06/01319

§ 371 Date: Mar. 31, 2008

(30) Foreign Application Priority Data
Mar. 3, 2005 (EP) 05251283.7
Dec. 9, 2005 (GB) 0525105.3

(51) Int. Cl. A61B 5/055

(52) U.S. Cl. 600/421

(57) ABSTRACT

In an electronic circuit and method for reducing interference in a measurement signal or signals, wherein the interference comprises a plurality of interference components: (a) There is at least one primary signal processing unit, each having a primary signal processing unit comprising a respective measurement signal input for receiving a respective one of said measurement signal or signals. The or each primary signal processing unit comprises a plurality of interference reduction modules. (b) A respective compensation signal component input is provided for each interference reduction module. (c) A compensation signal processing unit is provided, having a compensation signal input and comprising means for deriving from at least one compensation signal, a plurality of compensation signal components each of which is related to a respective one or more of the interference components. (d) A respective compensation signal component output is connected to a respective one of the compensation signal component inputs.

### FIELD OF THE INVENTION

This present invention relates to an electronic method and apparatus for reducing interference in a signal wherein the interference is of a large magnitude relative to the data component to be extracted from the signal. It is particularly, although not exclusively, suited to reducing noise in biopotential signal acquisition, which noise is caused by electrical and magnetic fields. It may also be used in other applications such as semiconductor physics, where electrical signals may be derived under conditions where a large noise component is present, e.g. due to a large varying magnetic field.

### BACKGROUND OF THE INVENTION

Functional magnetic resonance imaging (fMRI) is widely used in both medical and non-medical imaging to obtain a spatial image of “slices” through the brain. In the medical context, MRI is used to identify lesions such as areas of restricted blood flow or tumours. Outside the medical field, fMRI has, for example, been a useful tool in cognitive neuroscience for investigating brain response to various external stimuli.

Electroencephalography (EEG) has traditionally been used for investigations into brain activity. It may, for example, be used to investigate abnormal brain activity in disease states such as epilepsy or in certain psychiatric abnormalities.

If fMRI and EEG could be used together, they could advantageously combine both spatial and temporal information about brain function which would be of major benefit for both medical and non-medical uses. However, an EEG signal obtained from a scalp electrode is in the range typically of 10 μV to 100 μV at an impedance of around 500Ω to 50KΩ. The large magnetic and radio frequency (rf) fields produced by MRI machines swamp this signal with induced noise on the signal wire. In particular, switching of the MRI magnetic gradients causes extraneous pulses in the EEG signal.

However, at least two other sources of interference tend to occur in such a system. The first is powerline (mains) interference from the AC power system (typically 50 Hz or 60 Hz ). The second is ballistocardiogram (BCG) noise, ie noise caused by the pulsing blood flow of the subject interacting with the large static magnetic field of the MRI scanner.

Conventional known methods for rejecting interference in EEG include the use of a reference electrode and differential amplifier, electrical isolation of the EEG amplifiers, shielding of the electrode lead wires, driving the shield of the lead wires with a common mode voltage, and electrical filtering of the EEG signal. Additional strategies have been employed for EEG in fMRI, such as the use of carbon lead wires and inductors.

As will be explained further hereinbelow, the present invention is also useful in the application of medical or quasi-medical measurements, other than EEG.

For example, U.S. Pat. No. 5,445,162 proposes a system using electrodes and wiring designed to minimise noise pick-up and the fMRI and EEG data are obtained alternately. Thus, although the system purports to enable fMRI and EEG signals to be obtained at the same time from an individual, the technique does not permit obtaining truly simultaneous fMRI and EEG data. However, it does propose locating the EEG recording equipment outside the MRI room to minimise interference.

WO-A-03/073929 discusses the potential problems associated with concurrent fMRI and EEG measurements, namely noise induced in the EEG signal by the rf and magnetic fields (as mentioned above) and the disruption to the fMRI measurement by introduction of ferromagnetic material in the EEG electrodes, into the bore of the fMRI machine. This reference comments upon possibilities for alleviating these problems. One is to dispense with ferromagnetic materials in the EEG electrodes and to use an alternative such as carbon fibre. Another is to rearrange the EEG leads to minimise interference with the rf field.

The aforementioned WO-A-03/073929 also recognises safety problems inherent in deploying EEG equipment inside a pulsed rf field, eg due to induced currents. Solutions to these problems have included raising the impedance of the EEG detection circuit by means of resistors or by using different electrode systems or different electrode materials, or by incorporating a fibre optic link in the line between the electrodes and the circuit. The reference proposes that a better method of avoiding such hazards is to incorporate an amplifier within the electrode structure.

Despite these numerous proposals, there still remains a need for a system whereby truly simultaneous derivation of EEG and fMRI signals could be made possible, by eliminating the several major sources of interference on the EEG signal at an early stage in the processing circuitry rather than removing it by post-processing.

In principle, any one of a number of electrophysiological measurement systems can be combined with fMRI, instead of or in addition to EEG. Examples of these are electrocardiography (ECG), electromyography (EMG), electro-oculography (EOG), electroretinography (ERG) and galvanic skin response measurement (GSR). The same problems can occur with any electrophysiological measurement such as these, when used in combination with MRI, for example fMRI. Therefore, there is a need to suppress interference sufficiently when simultaneously conducting any electrophysiological measurement in combination with fMRI. For convenience, for the generic term electrophysiological measurement, hereinafter the abbreviation EPM will be used. The present invention is useful with any of these, or other EPM systems. It is also useful in other combinations of an EPM with interventions which utilise a large magnetic field, for example, transcranial magnetic stimulation (TMS).

We have now devised an electronic noise reduction circuit and method which solve this problem. In addition, in preferred applications, the present invention provides for substantially simultaneous data acquisition and read-out, thus providing minimal lag between data acquisition and data availability, as may otherwise arise due to post-processing, for example.

The electronic circuit and interference reduction method of the present invention may be employed with any measurement signal subject to interference but especially for any EPM alone or in combination with MRI, fMRI or TMS. It can also be used to reduce interference on signals obtained from magnetoencephalography (MEG). MEG is a technique analogous to EEG instead of using an electrode on the surface or the head, it uses an array of sensors to measure change In magnetic fields outside the skull, generated by neuronal activity.

### DEFINITION OF THE INVENTION

A first aspect of the present invention now provides an electronic circuit for reducing interference in a measurement signal or signals, wherein the interference comprises a plurality of interference components, the electronic circuit comprising:
(a) at least one primary signal processing unit, the or each primary signal processing unit having a respective measurement signal input for receiving a respective one of said measurement signal or signals and the or each primary signal processing unit comprising a plurality of interference reduction modules;
(b) a respective compensation signal component input for each interference reduction module;
(c) a compensation signal processing unit having at least one compensation signal input and comprising means for deriving from at least one compensation signal, a plurality of compensation signal components each of which is related to a respective one or more of the interference components; and
(d) the compensation signal processing unit also having a respective compensation signal component output for each compensation signal component, each said output being respectively connected to one of the compensation signal component inputs.

A second aspect of the present invention provides a method of reducing interference in a measurement signal or signals, wherein the interference comprises a plurality of interference components, the method comprising:
(a) inputting the at least one measurement signal to a respective primary signal processing unit, the or each primary signal processing unit comprising a plurality of interference reduction modules each having a compensation signal component input;
(b) inputting at least one compensation signal to a respective compensation signal processing unit wherein a plurality of compensation signal components are derived from the at least one compensation signal, each compensation signal component being related to a respective one or more of the interference components; and
(c) inputting the compensation signal components to respective compensation signal component inputs of the at least one primary signal processing unit.

The compensation signal is preferably derived from a separate compensation signal electrode connected to a neutral (relatively low in EEG content) part of the subject.

Preferably, the or each measurement signal is derived via a respective measurement signal line connected to its own measurement signal electrode. Preferably also, for each such measurement signal line, there is a corresponding reference signal line in close proximity therewith for a substantial part of their mutual lengths (or one or more group(s) of measurement signal lines may share a single reference signal line in close proximity in the same way). Each such reference signal line is connected to a respective reference signal electrode or connection point which in use, is positioned close to its corresponding measurement signal electrode. Preferably, the compensation signal line is also provided with a corresponding reference signal line connected to a reference signal electrode or connection point, situated close to the compensation signal electrode. Preferably, each reference signal is at least partially subtracted from the corresponding measurement signal, or signals in the case of a shared reference signal line, (or the compensation signal, as the case may be), for example with the respective primary signal unit (or compensation signal unit). Preferably, the compensation signal line has its own reference line in close physical proximity therewith along a substantial part of their mutual lengths.

For at least some measurement signal lines and/or the compensation signal line, more than one additional reference line may be provided, connected to the same reference electrode or its own respective reference electrode.

Preferably, at least one ground connection is provided between the subject and circuit ground in any apparatus according to the invention. This may be provided by one or more ground lines. A single ground electrode, for example of the same construction as a measurement signal electrode, may be situated at a position on the subject where electrophysical signals are absent or of low magnitude, such as the nape of the neck. However, a plurality of ground electrodes may be provided. When there is a plurality of ground lines, they may all be connected to a single ground electrode, or to respective dedicated ground electrodes. Alternatively, groups of ground electrodes may be connected to respective common ground electrodes. For example, separate respective ground lines may be provided for each signal, compensation, and reference connections or electrodes and lines, or each signal line/reference line pair and the compensation line/reference line pair shares a respective single common ground line. A ground line may also be provided for the compensation signal line and any accompanying reference line. In a one embodiment employing a plurality of such ground lines, substantially all of them are connected to a shared single ground electrode.

The interference reduction may optionally employ adaptive noise cancellation, preferably in real time, in which the amount of interference to be removed may be determined dynamically and varied over time.

Preferably, the interference reduction modules in each primary signal processing unit are arranged in series. Preferably, in each primary signal processing unit, separate interference reduction modules are provided for reducing at least two of magnetic switching interference, mains power interference, electrode/lead movement, eyeblink artifact interference and ballistocardiogram interference.

When the at least one compensation signal comprises two or more compensation signals these may be obtained from respective compensation signal electrodes, any or all of which may have the same form of construction as each other, or any or all of which may differ from each other. For example an eye blink compensation signal may be obtained from an EMG electrode which detects a physiological signal from muscle in the vicinity of the eyelid. A BCG compensation electrode may be obtained from an EEG type electrode positioned over an artery in the head. When a single electrode produces an output which combines more than one interference component in a single compensation signal, then circuitry in the compensation signal processing unit can filter the signal to extract the relevant interference components separately. Thus, where two or more compensation signals are utilised, preferably they are received via their own respective compensation signal input. Any reference herein to a, or the, compensation signal optionally includes reference to any or all of a plurality of compensation signals, where there is a plurality of such signals, unless the context forbids.

In an EEG measurement employing the present invention, any electrodes to the human or animal skin (eg scalp) may be dry or “wet” (i.e. employing an electrically conductive gel or paste).

A third aspect of the present invention provides an electronic circuit for reducing interference in a desired signal, the apparatus comprising
(a) at least one measurement signal line connected to a measurement signal electrode; and
(b) for each measurement signal line and measurement signal electrode connected thereto (or for each group of such measurement signal lines), a corresponding reference line connected to a reference electrode;
   the or each of said measurement signal lines (or group of measurement signal lines) being associated by being in close physical proximity with a respective one of the or each reference lines for a substantial part of their lengths, so that the or each measurement signal line with its corresponding reference line forms a measurement signal line (or measurement signal line group)/reference line pair, said electronic apparatus further comprising subtraction means for subtracting at least part of a signal on the or each reference line from the signal on the associated measurement signal line (or from respective signals of the measurement signal line group) in that measurement signal line (or measurement signal line group)/reference line pair.

A fourth aspect of the present invention provides a method of reducing interference from a desired signal, the method comprising
(a) providing at least one measurement signal line carrying a measurement signal and an interference signal;
(b) providing for each the or each measurement signal line (or group of signal lines), an associated reference line carrying at least an interference signal, said the or each measurement signal line (or measurement signal line group) and associated reference line being in close physical proximity for a substantial part of their lengths; and
(c) a subtraction step of subtracting at least part of a signal on the or each reference line from the signal on the or each associated measurement signal line (or from respective signals of the measurement signal line group) in that measurement signal line (or measurement signal line group)/reference line pair.

Regarding the third and fourth aspects of the invention, preferably a compensation signal line and most preferably, also an associated reference line are provided. As a generality, a compensation signal on the compensation signal line, derived from a separate compensation line electrode, is used to reduce interference in the or each measurement signal. Preferably, the signal on the compensation signal line is processed in a compensation signal processing unit to produce a plurality of compensation signal components. The compensation signal components are respectively used to reduce interference in respective interference reduction modules which process the respective measurement signal or signals preferably after subtraction of all or part of the corresponding reference signal or signals.

Thus one preferred class of embodiments combines the principles of the circuits of the first and third aspects of the present invention and the methods of the second and fourth aspects of the invention.

Any circuit element or method step independently may be implemented by analog or digital means.

### FURTHER ASPECTS OF THE INVENTION

The present invention may also be defined by any of the following further aspects of the invention A to I as set-out below. Each of these may optionally also employ any essential, preferred or optional feature of any other such aspects of the invention (method or apparatus as appropriate), and/or any other essential, preferred or optional feature of any other aspect of the invention described, defined or claimed elsewhere in this specification, including in terms of any measurements, types of applications and/or use of specific electrode arrangements or electrode support apparatus.

A. A method of reducing interference in a measurement signal, the method comprising:
(a) deriving a compensation signal;
(b) generating a plurality of compensation signal components from said compensation signal; and
(c) separately subtracting at least part of each of said compensation signal components from said measurement signal.

In this context, reference to separate subtraction means temporally sequential subtraction and/or by implementation in terms of respective electronic subtraction modules arranged in series, or else by implementation in terms of respective electronic subtraction modules in parallel. However, in the case of such subtraction modules used in parallel, one or more additional subtraction modules may also be arranged in series therewith. However, the above method may also be effected in whole or in part by hard wired digital components and/or appropriate software in a computer, the measurement signal and compensation signal having first been subjected to A/D conversion, optionally after preamplification to improve the signal to noise ratio.

The above method may also be used to reduce interference in a plurality of measurement signals using one or more compensation signals.

B. An electronic apparatus for reducing interference in a desired signal, the apparatus comprising
(c) a signal line connected to a signal electrode; and
(d) a reference line connected to a reference electrode;
   said signal line and reference line being associated by being in close physical proximity for a substantial part of their lengths, said electronic apparatus further comprising subtraction means for subtracting an interference signal on the reference line from an interference signal on the signal line thereby to enhance a desired signal on the signal line.

C. An electronic apparatus for reducing interference in a desired signal, the apparatus comprising:
(a) a plurality of signal lines, each connected to a respective signal electrode; and
(b) one or more reference lines, each connected to respective one or more reference electrodes;
   each of said signal lines (or group of said signal lines) being associated by being in close physical proximity with a respective one of said reference lines for a substantial part of their lengths, so that each signal line (or signal line group) with its corresponding reference line forms a signal line (or signal line group)/reference line pair, said electronic apparatus further comprising subtraction means for subtracting an interference on each reference line from an interference signal on the associated signal line (or from each signal line in that signal line group) in that signal line (or signal line group)/reference line pair.

D. A method of reducing interference from a desired signal, the method comprising
(a) providing a signal line carrying a desired signal and an interference signal;
(b) providing a reference line carrying at least an interference signal, said signal line and reference line being associated by being in close physical proximity for a substantial part of their lengths; and
(c) a subtraction step of subtracting the interference signal on the reference line from the interference signal on the signal line.

E. A method of reducing interference from a desired signal, the method comprising
(a) providing a plurality of signal lines, each carrying a desired signal and an interference signal;
(b) providing one or more reference lines, each carrying at least an interference signal, each signal line (or group of signal lines) being associated by being in close physical proximity for a substantial part of its length with a respective reference line to provide respective signal line/reference line pairs; and
(c) performing a subtraction step of subtracting the interference signal on each respective reference line from the interference signal on the associated signal line (or from each signal line in that signal line group) of its signal line (or signal line group)/reference line pair.

F. An electronic apparatus for reducing interference in a signal derived from an EPM the apparatus comprising
(a) a signal line connected to a signal electrode;
(b) a reference line connected to a reference electrode; and
(c) at least one ground line for said signal line and reference line, said ground line or lines being connected to at least one ground electrode or individually to respective ground electrodes;
   said electronic apparatus further comprising subtraction means for subtracting an interference signal on the reference line from a signal on the signal line.

G. An electronic apparatus for reducing interference in a desired signal, the apparatus comprising:
(a) a plurality of signal lines, each connected to a respective signal electrode; and
(b) one or more reference lines connected to one or more reference electrodes; and;
(c) one or more ground lines connected to one or more ground electrodes;
   said electronic apparatus further comprising subtraction means for subtracting an interference signal on the or each reference line from an interference signal on the signal lines and/or subtracting an interference signal on the or each ground line from the interference signal on the signal lines.

H. A method of reducing interference on a signal derived from an EPM, the method comprising
(a) providing a signal line carrying a desired signal and a first interference signal, said signal line being connected to a signal electrode;
(b) providing a reference line carrying at least a second interference signal, said reference line being connected to a reference electrode;
(c) providing a ground line for said signal line and reference line, said ground line or lines being connected to at least one ground electrode or individually to respective ground electrodes; and
(d) a subtraction step of subtracting the second interference signal on the reference line from the first interference signal on the signal line.

I. A method of reducing interference from a desired signal, the method comprising
(a) providing a plurality of signal lines, each carrying a desired signal and a first interference signal;
(b) providing one or more reference lines carrying at least a second interference signal;
(c) providing one or more ground lines; and
(d) performing a subtraction step of subtracting the second interference signal from said first interference signal.

In any apparatus or method according to aspects B to I of the present invention, at least one compensation signal line may be provided for connection to a compensation signal electrode. The compensation signal electrode is preferably located on a subject in a “neutral” position (eg in the case of EEG, on or near an ear). The resultant at least one compensation signal, delivered via the compensation signal line(s) may be used to at least partially reduce interference on the (measurement) signal line or lines, eg by a subtractive process. The compensation signal line is preferably associated with its own reference line which is preferably in close physical proximity thereto along a substantial part of their mutual lengths and is connected to a reference electrode (node) associated with the compensation signal electrode.

### DETAILED DESCRIPTION OF THE INVENTION

In accordance with the third and fourth aspects of the invention (which optionally may also incorporate the features of the first and second aspects of the invention, respectively), a “reference loop” is used for subtracting at least some interference signals induced by external fields into a circuit loop. In preferred embodiments described hereinbelow, this circuit loop is formed by the connection between the living body and electronic amplification circuitry. In the described embodiments, a simplified version of the reference loop is described for use in multi-channel EPM recordings, such as EEG recordings in order to reduce noise voltages induced by the magnetic fields generated in a functional magnetic resonance imaging device (fMRI). In addition, an embodiment of a complete circuit means is described for acquiring simultaneous EPM in the MRI or fMRI environment, with minimal interference to the EPM and fMRI. EPM signals such as EEG signals can still have large interference components if used also without fMRI or the like, eg generated by electric motors in the vicinity. The present invention is also useful in such applications, reducing or removing the need for screening of the noise source and/or data acquisition circuitry.

In order to achieve EPM data acquisition, concurrent with fMRI, the EPM data acquisition circuitry must reject interference caused by external (to the body) electric and magnetic fields. The main sources of interference are low frequency electric and magnetic fields from the AC power mains (commonly 50 or 60 Hz), switched magnetic fields from fMRI with fundamental frequencies ranging down to approximately 500 Hz, and radio frequency (rf) electromagnetic fields from fMRI ranging from 60 to 130 MHz. Another source of interference is ballistocardiogram noise due to pulsing of circulatory blood in the magnetic field. In addition, the large static magnetic field of the MRI scanner causes interference voltage to be induced in EPM signal lines whenever movement of the electrodes or lead wires occurs. At least two of these are reduced as separate interference components in accordance with the first and second aspects of the present invention.

In the broadest aspect, the third and fourth aspects (and preferred embodiments of the first and second aspects) of the present invention utilise a single signal line and a single reference line. However, most practical applications will involve use of a plurality of signal lines with associated reference lines. The single signal line can be connected to a respective separate signal electrode. The reference lines may be connected to a single reference electrode or to a respective separate reference electrode or any other arrangement involving multiple reference electrodes.

Each signal line (or group of signal lines) may therefore be associated with a corresponding one of the reference lines to be in close proximity for a substantial part of their lengths, so that each respective signal line and associated reference line constitutes a respective signal line (or signal line group)/reference line pair. The subtraction means is then arranged to subtract an interference signal on each reference line from the interference signal on its associated signal line (or each signal line of the respective group) in the pair, thereby enhancing the desired signal on that signal line.

Any reference line is preferably connected to a conductive member physically close to, but not in direct electrical contact with part of the human or animal body (eg the scalp in the case of an EEG measurement). This conductive member may, for example, be in the form of a conductive mesh. In other embodiments, the reference lines may be in direct electrical connection with the subject, eg in the case of EEG to a signal electrode which may, for example be in contact with an earlobe or with skin close to an ear.

Essential for some, whilst merely preferable for other aspects of the present invention is provision of one or more ground lines. Any signal line/reference line pair may share a common ground line, preferably in close physical proximity with both, or each signal line and reference line may be provided with its own ground line, preferably in close physical proximity therewith. A combination of such arrangements is also possible (one or more shared ground lines for some signal/reference line pairs and one or more individual ground lines for any one or more others). All ground lines may be connected to a common ground electrode or to individual respective ground electrodes, or any other arrangements involving multiple ground electrodes. Preferably, the or each ground electrode is in direct (low resistance) contact with the subject (eg the skin of the head or scalp in the case of EEG), as described further hereinbelow. In an especially preferred class of embodiments, a plurality of measurement signal lines has each connected to a respective measurement signal electrode. Each measurement signal line (or group of measurement signal lines) has its own associated reference signal line connected to a respective reference signal electrode (node). A separate ground electrode is connected to a ground line and a separate compensation signal electrode is connected to a compensation signal line. The compensation signal line and ground line each have a respective associated reference line connected to a dedicated additional respective reference electrode.

Where an individual line or lines (measurement signal, compensation signal, reference signal or ground) is or are connected to its, or their, own dedicated electrode (signal, reference, or ground, respectively), that electrode may be embodied as two or more electrode entities with the reference line or lines being connected thereto in parallel. The terms “electrode” and “node” (see below) are to be interpreted as encompassing these possibilities, except where explicitly stated to the contrary or where the context forbids.

The or each measurement signal line, compensation signal line and/or ground line, as the case may be, may be in close physical proximity for a substantial part of the length thereof, with a respective reference line, a respective ground line, or both, preferably twisted together therewith.

Preferably, signal and any ground electrodes are in direct electrical connection with the subject (usually the head, or head/neck region when the EPM is EEG, e.g. mainly to the scalp). This preferably means an individual electrode contact resistance of less than 1 Kohms. However, reference electrodes are preferably not in direct electrical contact with the subject but are electrodes in close physical proximity with the subject, preferably each respectively close to its associated signal electrode.

Preferably, and particularly when the EPM is EEG the reference electrodes are arranged as a mesh. Then signal and reference electrodes may be arranged over the head or scalp but one signal/reference electrode pair may be attached to positions where the pick-up of physiological electrical signals will be low, such as beneath the ear. Thus, it is to be understood that the term “electrode” includes variants which are not in direct electrical contact with the subject.

A preferred form of construction comprises a flexible, electrically conductive elastic reference mesh material acting as a cap to hold the electrodes in place. The reference mesh material may be coated with an insulating layer to electrically isolate the mesh from the body and electrodes. All components are preferably made from materials chosen to be resistant to chemical disinfectants and detergents.

Another aspect of the present invention provides an electrode support structure apparatus for effecting an EPM, the apparatus comprising an electrode support having supported thereon, an array of measurement signal electrodes presented for contacting the skin of a subject, first connection means being provided for independent electrical connection to each of said measurement signal electrodes, the apparatus further comprising an electrically conductive mesh having one or more of reference nodes and second connection means for independent electrical connection to the or each of said reference nodes. This support structure may be employed with any circuit, method or apparatus according to any other aspect of the present invention.

As used herein, any electrical contact point to a reference mesh is usually termed an “electrode”. However, the term “node” is also used for such a contact point with a reference mesh and as such, can be considered synonymous with electrode, whether or not any part of the mesh is in direct electrical contact with the subject, eg with the skin of the subject.

One suitable form of construction is in the form of a rigid or flexible cap, preferably having two layers of insulating elastic cap material with an electrically conductive reference mesh construction (preferably flexible) sandwiched between, and electrodes anchored to the cap. Cap structures for supporting EEG electrodes are already known from WO-A-00/27279 and U.S. Pat. No. 6,708,051.

Each electrode site on any suitable cap structure, may for example have four wires—two for the signal loop and two for the reference loop—arriving as two twisted pairs twisted around each other. One wire connects to the body electrode; one wire connects to the reference mesh next to the electrode; one wire proceeds across the cap to the body ground electrode; and one wire proceeds across the cap to the reference mesh ground connection. A multi-channel arrangement would comprise a plurality (n) of such sites.

Reference mesh material can be made of carbon loaded fabrics, foam or yarns (carbon wire). Other conductive materials can be used for loading in addition to or in lieu of carbon, such as a silver-coated polymer substrate, eg nylon.

For the avoidance of doubt, reference to subtraction in accordance with any aspect of the present invention means any attenuation of interference on a signal line by deriving an interference signal from a corresponding reference line and using it to diminish the interference signal on the signal line. Arithmetic subtraction as well as other operations are included within this term. The definition includes substantial total elimination of the interference signal but also covers at least some diminution of the interference signal from the signal line.

Reference herein to any two or more lines being associated in close proximity for a substantial part of their length(s) preferably means that the respective lines run in close physical proximity for at least 50%, more preferably at least 60%, still more preferably at least 70%, yet more preferably still at least 80% and most preferably at least 90% of their lengths (when one or more wires is longer than any other relevant wire, then these percentages are of the longest).

Any lines which are in close proximity may be arranged thus by any suitable means, eg coaxially (such as with the reference line surrounding a core of the signal line, or vice versa) or by being run together as a twin wire pair (or multi-wire group) or by any other means, but most preferably, by being twisted together.

The subtraction means preferably comprises a differential amplifier with inverting and non-inverting inputs connected to signal line(s) and reference line(s) respectively.

Each signal line/reference line pair may be shielded, for example by a metallic sheathing which suitably may be connected to a ground connection.

The subtraction means may also comprise one or more common mode chokes associated with the respective signal line/reference line pairs, the windings of each such common mode choke being connected to a respective one of the signal line and the reference line. The subtraction means preferably also comprises low pass filter means, especially a seventh order low pass filter, an exemplary embodiment of which comprises a 0.05° Equiripple-type filter.

The apparatus and method of any aspect of the present invention may be deployed in the MRI room itself, although recording may be conducted outside that room. The apparatus of any aspect of the present invention may be substantially totally electrically wired, ie not require any optical or wireless link, although the latter are also possible.

The present invention will now be explained in more detail by way of the following description of preferred embodiments, and with reference to the accompanying drawings, in which:

### DESCRIPTION OF THE DRAWINGS

FIG. 1 shows a schematic of an EEG and fMRI system, in which an interference reduction apparatus and method according to the present invention may be employed;

FIG. 2 shows the fMRI pulse sequence employed in the set-up of FIG. 1;

FIG. 3 shows a front-end circuit for use with the EEG system of FIG. 1;

FIG. 4 shows a downstream circuit for use with the front-end circuit of FIG. 3;

FIG. 5 shows a perspective view of an electrode cap according to, and for use in, the present invention; and

FIG. 6 shows a cross section through one electrode region of the electrode cap shown in FIG. 5.

### DESCRIPTION OF PREFERRED EMBODIMENTS

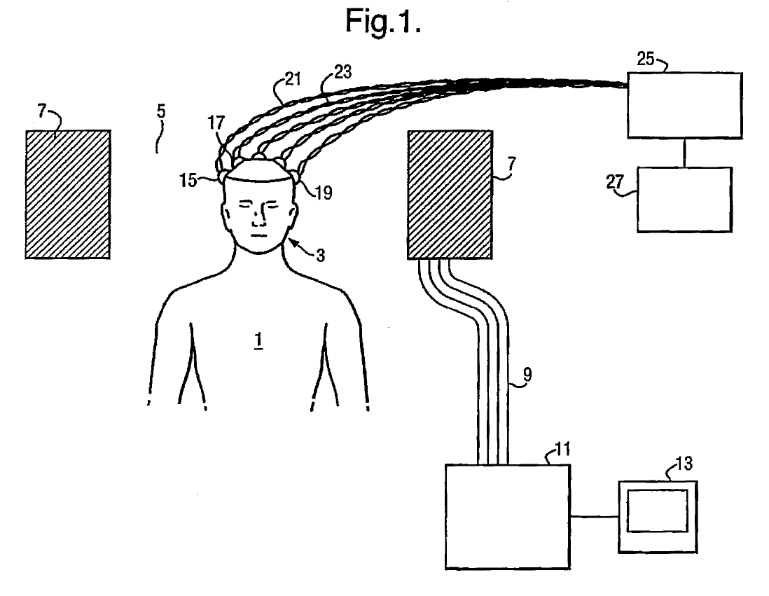

In the embodiments of FIGS. 1-5, signal and reference lines are in close physical proximity along substantial parts of their mutual lengths. Reference signals on the reference lines are at least partly subtracted from the respective measurement signals on their associated measurement signal lines to help reduce interference. Thus, these embodiments are in accordance with the third and fourth aspects of the present invention.

FIG. 1 shows a basic fMRI and EEG system in which the apparatus and method of the present invention may be employed.

As shown in FIG. 1, a subject 1 is arranged with the subject's head 3 located within the bore 5 of an fMRI coil unit 7 which carries the magnetic field windings and rf coils. These coils and windings are energised via a multiplicity of wiring connections 9 etc which connect the coil unit 7 to operational circuitry 11. The operational circuitry unit is connected to a memory and display unit 13 whereby the MRI scans can be stored, displayed and printed at will.

A plurality of electrodes 15, 17 etc for obtaining EEG signals are attached to the scalp of the subject 1. As will be explained in more detail hereinbelow, one of these electrodes 19 is a “reference electrode”. Signals from the electrodes 15, 17, 19 etc are conveyed by wires 21, 23 etc to an EEG control unit 25 which is connected to a recorder 27 situated outside the MRI room.

The combined fMRI/EEG arrangement may be considered to apply to any specific embodiment of EEG processing circuitry described hereinbelow.

In a worked embodiment, used for obtaining data presented in more detail hereinbelow, the MRI system was the Siemens Allegra™ (3.0T)-MR6.

The Siemens Allegra™ 3T is a head-only research magnet. It has the necessary hardware and software to perform basic and clinical scans. Gradient hardware consists of a 36 cm I.D. asymmetric gradient coil capable of imaging at 60 mT/m with slew rates in excess of 600 T/m/s at a duty cycle of 70% allowing single shot echoplanar imaging (EPI) at a sustained rate of 14 images/second. The system has a 15 kW RF amplifier, and 8 RF preamp channels for this system supports the Syngo™ software on a Windows™ NT platform.

The EPI regime employed 1 to 8 gradient switching pulses (images) per second. Gradient strength: 20-35 mT/m, max 40 mT/m; Slew rate: 400 mT/m/msec. Pulse width: 0.32-0.64 msec, oscillating between positive and negative gradients. Rf pulse freq: 126 MHz, frequency modulated for slice position.

The conventional sequence used for fMRI is multi-slice echo planar imaging. In this, the largest gradient is applied as a bi-polar square wave, which is often modified to be more trapezoidal or sinusoidal in form (to smooth the edges). Typically for one image this is applied for 20-100 ms with a fundamental frequency of 2 to 0.5 kHz. One of the other two gradients is usually applied as a series of smaller pulses (100 μs duration typical) at the zero crossings of the big switched gradient, whilst the third (slice select) gradient is generally just applied at the beginning of the sequence as a bi-polar square pulse, typically lasting 3-5 ms. The rf is usually just applied at the same time as the slice select gradient.

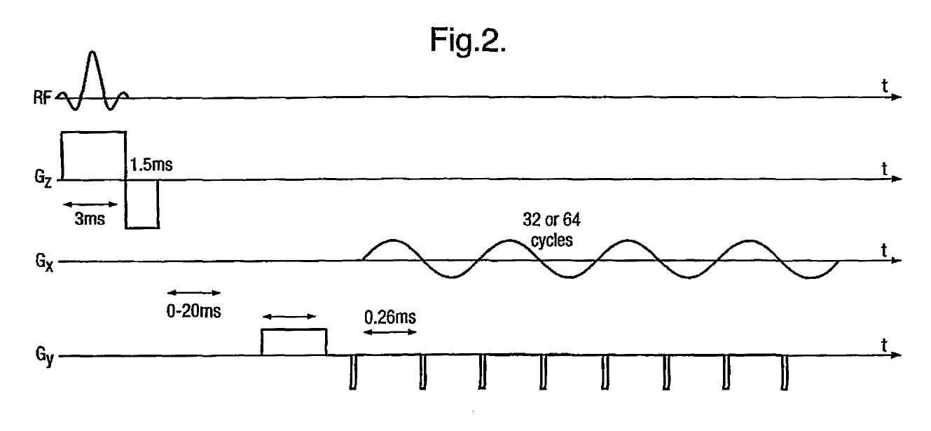

FIG. 2 shows the basic EPI sequence used. Gz denotes slice select, Gx is the large gradient and Gy is the smaller pulsed gradient. The rf pulses are also shown. In the tests described further hereinbelow, Gx was on for 30 ms. Depending on the MRI machine used, slice gradient times can vary by a factor of 2, and the switched gradient could be lower by a factor of 2 in frequency and strength.

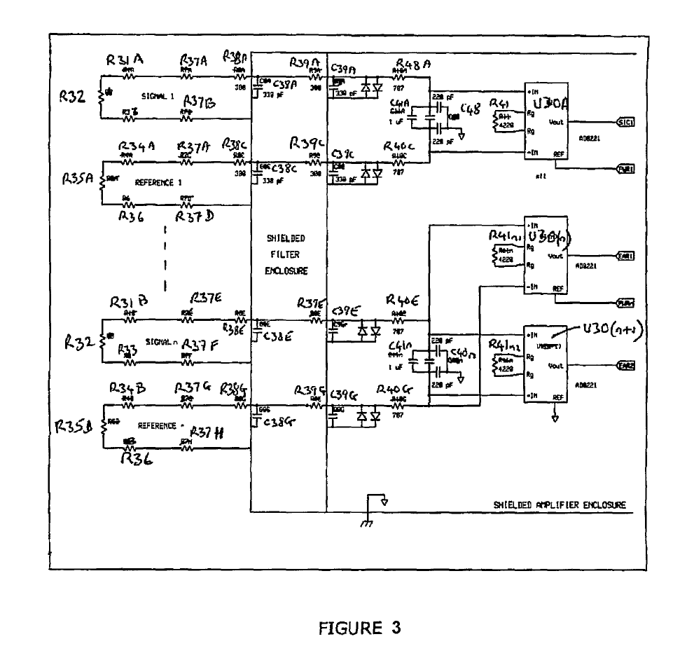

Referring to FIG. 3, there is shown a circuit for processing the EEG signals. Shown are n measurement channels, where n ranges typically from 2 to 1024. For convenience, only the 1^{st} and n'th channels are actually shown in the drawing. Each measurement channel comprises a signal line and a reference line. The signal line and reference line of each channel are paired with a respective ground line.

Thus, as shown, there are n measurement channels (1 to n) of identical construction such as is shown for measurement channel 1. As the n channels are of identical construction, only Channel 1 will be described in detail below. Channel 1 comprises signal line pair designated “Signal 1” and reference line pair “Reference 1”. As depicted, the signal line of “Signal 1”, is connected to the scalp for EEG via a signal or measurement electrode with an impedance represented by resistor R31A, preferably having an electrode impedance of around 10K ohms or less. Other signal electrodes are denoted R31B etc. All body electrodes preferably are constructed of a resistive material such as carbon-loaded plastic, or the bare ends of carbon wire. Contact to the body is made via a conductive paste.

In a signal channel 1, outside a shielded filter enclosure, a number of resistors R32, R31A, R37A, R38A, R33, and R37B are connected in series. A first terminal of the resistor R32 is connected to a first terminal of the resistor R31A and the second terminal of the resistor R31A is connected to the first terminal of the resistor R37A, the second terminal of the further resistor R37A being connected to the first terminal of the resistor R38A. The second terminal of the resistor R32 is connected to the first terminal of the resistor R33 and the second terminal of the resistor R33 is connected to the first terminal of the resistor R37B, the second terminal of the resistor R37B being terminated on the shielded enclosure which is connected to circuit ground. In the reference channel 1, outside a shielded filter enclosure, a number of resistors R35A, R34A, R37C, R38C, R36 and R37D are connected in series. The first terminal of a first resistor R35A is connected to the first terminal of the resistor R34A, the second terminal of the resistor R34A being connected to the first terminal of the resistor R37C. The second terminal of the further resistor R37C is connected to the first terminal of the resistor R38C. The second terminal of the resistor R35A is connected to the first terminal of the resistor R36 and the second terminal of the resistor R36 is connected to the first terminal of the resistor R37D, the second terminal of the resistor R37D being terminated on the shielded enclosure which is connected to circuit ground.

Similar connections exist for the other channel/reference pairs.

For channel 1 (and similarly for all signal channels), the wires represented by R37A and R37B are twisted together tightly to minimize the loop area formed by the wires and hence minimize induced magnetic field interference in the signal.

In measurement channel 1, R34A is a connection of a carbon wire to a conductive reference mesh that spans the surface of the head but is not in electrical contact with the body. R34A is located very close to R31A. R35A represents the impedance of the reference mesh. The wires for the reference loop (R37C and R37D) are twisted together tightly to minimize loop area, and the pair is twisted together with the R37A-R37B pair to match the paths followed by the loops.

Preferably the impedances of R31A and R34A are matched, as well as those of R32 with R35A, and R33 with R36. However, it is acceptable if only the sums of impedances R31A+R32+R33 and R34A+R35A+R36 are reasonably matched.

Each resistor designated R32 represents the impedance of body tissue, typically 100 ohms, between signal and ground electrodes. Each resistor designated R33 represents the ground electrode, preferably 10K ohms or less, located typically at the base of the neck. Similarly, each resistance R36 represents the corresponding ground electrode for the associated reference electrodes R34A, R34B etc. Resistors R37 (A through H) represent the resistance of the carbon wire connecting the electrode or reference loop to the electronic amplifiers, combined with the resistance of a patient safety resistor. A typical value for R37 is 13K ohms. The safety resistor typically is 12.5K ohms (range 10K to 15K ohms), preferably non-magnetic (such as Ohmite Macrochip™ SMD resistor), and is mounted in the electrode wire close (within 0.3 m) to the patient.

All of the components associated with the reference mesh and body electrodes may be considered impedances (i.e. having to greater or lesser degrees, resistive, inductive and capacitive components). Thus, except where indicated explicitly to the contrary or where the context does not permit, as used herein, all references to resistance may be regarded as including reference to impedance and “resistive” should be interpreted likewise.

The body electrodes (R31A-etc) are composed of resistive elements at all frequencies and significant capacitive elements down to about 10 Hz. R32, the body tissue beneath the scalp, may be considered to be solely resistive below 100 Hz. R34A-etc in the reference mesh corresponds to R31A-etc, and R35A-etc in the reference mesh corresponds to R32, with the goal being to match these corresponding elements electrically, primarily in the frequency range for physiological signals of interest, 1-1000 Hz. Above that range the electronic filters take over for eliminating magnetic and rf noise. There are capacitive and inductive elements in the reference mesh that are significant at rf, and matching the impedances of the loops at rf is desirable. However, for matching purposes, the maximum tolerable range may be considered to be a DC resistance measured in a reference mesh loop of 50 to 50K ohms (measured at the point where the loop connects to the cable, for example, at the connection of resistance R37C with R34A). A preferred range would be an impedance of between 1K and 10K ohms measured in the reference loop at a frequency of 10 Hz. The body electrode impedances (at 10 Hz) are preferably lower than 10K ohms with a maximum of 20K ohms measured between the signal electrode and ground electrode.

Generally, there may be some level of electrical inter-connection between the points of connection to the reference mesh, depending on the construction. If a continuous conductive fabric or foam is used, there is significant connection throughout the material, and R35A-etc are all connected by primarily resistive and capacitive elements. At the other end of the spectrum, if a lattice network is used, then conductive strings connect the various junctions where R35A-etc. meet R34A-etc. Thus, “reference electrode” is to be interpreted as encompassing the extremes and all possible intermediate forms of construction. The connections are again primarily resistive and capacitive, and can be every junction connected to every other junction at one extreme, or at the other extreme just nearest neighbouring junctions connected.

The nth channel is connected to a neutral location (close to areas of physiological signals of interest but without signal activity) such as behind the ear or on the earlobe for EEG, and has the same configuration (as the signal channels) of a signal loop paired with a matching reference loop. Thus, the n'th channel conveys a compensation signal whilst measurement signals are provided via channels 1 to (n−1). R33 serves as a common ground electrode to the body for all signal circuits, and similarly R36 is a common ground connection to the reference mesh for all the reference circuits.

The patient cable consisting of all carbon wires twisted in pairs is approximately 2 to 5 meters in length and terminates at the shielded enclosure containing rf filters, analog amplifiers, filters, A/D converters and digital control circuitry. Filtering for rf interference is accomplished with two layers of filters separated by a five-sided shielded enclosure (labelled “Outer Shielded Filter Enclosure” in FIG. 3). The first rf filter begins with resistors R38, 100 to 1K ohms, carbon or thick film composition. Capacitors C38 represent feedthrough capacitors of 1000 pF to 10,000 pF inserted into the wall of the shielded filter enclosure. Alternatively, capacitors C38 may be replaced by a filter connector such as Amphenol™ part number 21-474021-025 which has a pi filter configuration. The second rf filter begins with resistors R39 (same values and types as R38), with feedthrough capacitors C39 (same values and types as C38) inserted into the wall of the shielded amplifier enclosure. Further rf filtering may be accomplished with the use of a 2-channel common mode choke for the two leads of each channel, inserted in the lines after the second rf filter. The rf filters also include capacitors C40, which are X2Y components, in combination with resistors R40. In addition, reverse polarity diode pairs are connected to the signal and reference lines before resistors R40 to limit currents in the patient to IEC60601 safety standards in single fault conditions that may arise in the electronic circuitry.

In the signal channel 1 outside the shielded filter enclosure, the second terminal of resistor R38A is connected to the first terminal of feedthrough capacitor C38A. Inside the outer shielded filter enclosure, the second terminal of feedthrough capacitor C38A is connected to the first terminal of resistor R39A. The mounting terminal of feedthrough capacitor C38A is terminated on the wall of the outer shielded filter enclosure. The second terminal of resistor R39A is connected to the first terminal of feedthrough capacitor C39A. Inside the inner shielded filter enclosure, the second terminal of feedthrough capacitor C39A is connected to the first terminal of diode D1A, the first terminal of diode D2A, and the first terminal of resistor R40A. The mounting terminal of feedthrough capacitor C39A is terminated on the wall of the inner shielded filter enclosure. The second terminal of diode D1A and the second terminal of diode D2A are connected to circuit ground. The second terminal of resistor R40A is connected to the first terminal of X2Y capacitor C40A.

In the reference channel 1 outside the outer shielded filter enclosure, the second terminal of resistor R38C is connected to the first terminal of feedthrough capacitor C38C. Inside the outer shielded filter enclosure, the second terminal of feedthrough capacitor C38C is connected to the first terminal of resistor R39C. The mounting terminal of feedthrough capacitor C38C is terminated on the wall of the outer shielded filter enclosure. The second terminal of resistor R39C is connected to the first terminal of feedthrough capacitor C39C. Inside the inner shielded filter enclosure, the second terminal of feedthrough capacitor C39C is connected to the first terminal of diode D1C, the first terminal of diode D2C, and the first terminal of resistor R40C. The mounting terminal of feedthrough capacitor C39C is terminated on the wall of the inner shielded filter enclosure. The second terminal of diode D1C and the second terminal of diode D2C are connected to circuit ground. The second terminal of resistor R40C is connected to the second terminal of X2Y capacitor C40A.

Circuit power ground (common), denoted by the triangle symbol within the shielded amplifier enclosure near the bottom of FIG. 3, is preferably connected to the metallic shield enclosure in one location as shown in the Figure. Although circuit power connections are not shown in the Figures, it is understood that the analog integrated circuit components requiring power are connected to bipolar power supplies of typically ±2.5 volts to ±10 volts, and the digital integrated circuit components are connected to typically +3 to +5 volts. Power is supplied preferably from batteries located within the shielded amplifier enclosure, but may also be supplied from an external power source (isolated medical grade power supply or batteries) if the power inputs are filtered for rf at the shield enclosure, using filters similar to those shown for the signal lines.

U30A is an instrumentation amplifier that is configured to subtract the reference loop signal connected to the inverting input and also the powerline component of the compensation signal connected to the reference input. A preferred component for U30A is the AD8221 instrumentation amplifier manufactured by Analog Devices, Inc. This device maintains a very high common mode rejection at much higher frequencies than other commercially available instrumentation amplifiers, resulting in improved subtraction of high frequency noise components generated by fMRI magnetic field switching. Additionally, the AD8221 has high impedance inputs, thus allowing the direct connection of inputs from measurement and reference electrodes without the need for buffer amplifiers, as is shown in FIG. 3. However, if adjustment of gain in the reference signal is desired prior to the subtraction stage, buffer amplifiers with variable gain may be added prior to the inputs of amplifier U30A in FIG. 3. In the nth channel, the amplifiers corresponding to U30A are designated as U30(n) and U30(n+1) respectively.

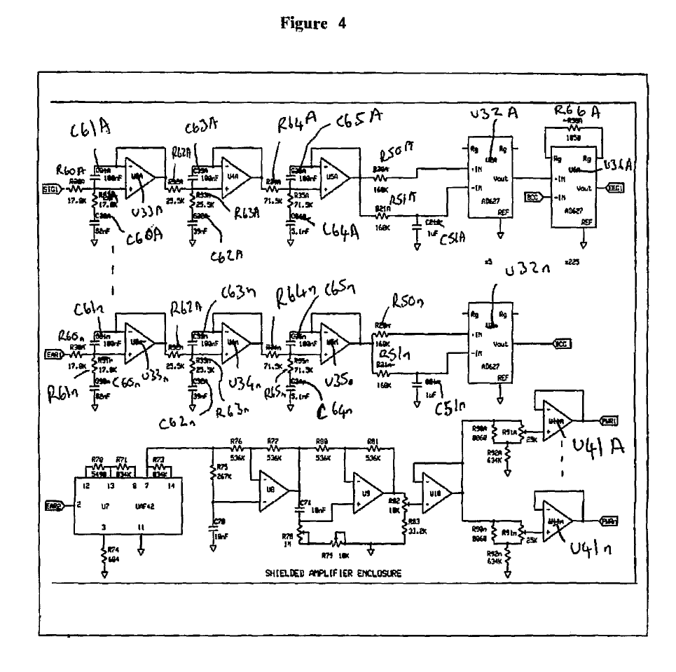

The compensation signal is derived from a neutral electrode location such as the earlobe or mastoid bone behind the ear in EEG. This signal has fMRI interference reduced by subtracting a reference loop signal as previously described. In FIG. 3, the compensation signal and its loop reference are connected to the non-inverting and inverting inputs, respectively, of both instrumentation amplifiers U30(n) and U30(n+1). The output of U30(n) is used to derive components of the compensation signal that are not related to powerline interference. As such, the reference input pin of U30(n) is connected to the powerline component derived from U30(n+1) in order to remove powerline interference. In contrast, the reference pin of U30(n+1) is connected to ground in order to maintain the powerline component. The powerline component is obtained by narrow bandpass filtering of the output of U30(n+1) at 50 or 60 Hz followed by phase and amplitude adjustment. In FIG. 4, the output of U30(n+1) (denoted as EAR2) is connected to bandpass filter U37 and operational amplifier U38-U40 and associated circuitry for phase and amplitude adjustment. The powerline component is a 50 or 60 Hz sine wave with −180 degree phase and amplitude matched to the powerline component present in each measurement signal channel. In order to closely match individual powerline amplitudes across signal channels, separate amplitude adjustments are provided (U41A through U41n and associated voltage dividers in FIG. 4) for each signal channel and the U30(n) compensation channel. Variable resistors R91 may be implemented as digitally-controlled potentiometers for dynamic adjustment of the powerline component amplitude. The powerline reference signals (PWR1 and PWRn in FIGS. 3 and 4) are fed back to the reference inputs of the AD8221 instrumentation amplifiers for each channel resulting in significant reduction of powerline interference. This approach eliminates an extra differential amplifier by accomplishing subtraction of both the reference loop signal and the powerline component of the compensation signal in one amplifier.

As shown in FIG. 4, SIG1 is a measurement signal with powerline and reference loop subtracted. SIG1 is fed into a 6-pole low pass Butterworth filter (U33 to U35 and associated circuitry) with cutoff frequency of 100 Hz to further reduce residual high frequency interference from fMRI sources. DC electrode potentials, BCG and other residual interference from fMRI below 100 Hz remain with the measurement signal at this stage. DC electrode potentials are removed with split low pass filters and differential amplifier (U36 and associated circuitry in FIG. 4) and the signal is amplified with a gain of 5.

Other components in the compensation signal such as BCG and residual fMRI noise sources are reduced by spitting off a second reference derived from the ear channel, beginning with U30(n) and EAR1 in FIG. 3. EAR1 has powerline interference removed as described above, and is then amplified and filtered (U33n-U35n and associated circuitry in FIG. 4) using the same method as used in the measurement signal channel. The resulting reference signal “BCG” is composed of BCG and residual fMRI interference, but not powerline. It is subtracted from each measurement signal channel in the final gain stage by means of a differential amplifier (AD627, U36A for SIG in FIG. 4). Although not shown in FIG. 4, individual adjustment of the BCG component for each measurement signal channel may be implemented with digitally-controlled potentiometers in a voltage divider configuration similar to the R91 and U41 combination used to adjust the amplitude of the powerline component in FIG. 4. The output of U36A, EEG1, is the measurement signal with interference removed by means of subtraction of each of a reference loop signal, a powerline component of the compensation signal, and a BCG/residual fMRI interference component of the compensation signal. Each of the interference components may be adjusted for gain separately from the others.

Thus, in measurement channel 1 the first terminal of the X2Y capacitor C40A is connected to the non-inverting terminal of instrumentation amplifier U30A. The second terminal of the X2Y capacitor C40A is connected to the inverting terminal of instrumentation amplifier U30A. Each of the terminals of resistor R41A are connected to a respective Rg terminal of instrumentation amplifier U30A. The output terminal of instrumentation amplifer U30A is connected to the first terminal of resistor R60A. The reference terminal of U30A is connected to the output terminal of operational amplifier U41A. In channel n, the first terminal of the X2Y capacitor C40n is connected to the non-inverting terminal of instrumentation amplifier U30n and the non-inverting terminal of instrumentation amplifier U30(n+1). The second terminal of the X2Y capacitor C40n is connected to the inverting terminal of U30n and the inverting terminal of U30(n+1). Each of the terminals of resistor R41B are connected to a respective Rg terminal of instrumentation amplifier U30n. The output terminal of U30n is connected to the first terminal of resistor R60n. The reference terminal of U30n is connected to the output terminal of operational amplifier U41n. Each of the terminals of resistor R41C are connected to a respective Rg terminal of U30(n+1). The output terminal U30(n+1) is connected to terminal 2 of filter module U37. The reference terminal of U30(n+1) is connected to circuit ground.

Continuing in measurement channel 1, the second terminal of resistor R60A is connected to the first terminal of capacitor C61A and the first terminal of resistor R61A. The second terminal of capacitor C61A is connected to the inverting input of operational amplifier U33A. The second terminal of resistor R61A is connected to the first terminal of capacitor C60A and the non-inverting input of operational amplifier U33A. The second terminal of capacitor C60A is connected to circuit ground. The output terminal of operational amplifier U33A is connected to the inverting input of operational amplifier U33A and the first terminal of resistor R62A. The second terminal of resistor R62A is connected to the first terminal of capacitor C63A and the first terminal of resistor R63A. The second terminal of capacitor C63A is connected to the inverting input of operational amplifier U34A.

The second terminal of resistor R63A is connected to the first terminal of capacitor C62A and the non-inverting input of operational amplifier U34A. The second terminal of capacitor C62A is connected to circuit ground. The output terminal of operational amplifier U34A is connected to the inverting input of operational amplifier U34A and the first terminal of resistor R64A. The second terminal of resistor R64A is connected to the first terminal of capacitor C65A and the first terminal of resistor R65A. The second terminal of capacitor C65A is connected to the inverting input of operational amplifier U35A. The second terminal of resistor R65A is connected to the first terminal of capacitor C64A and the non-inverting input of operational amplifier U35A. The second terminal of capacitor C64A is connected to circuit ground. The output terminal of operational amplifier U35A is connected to the inverting input of operational amplifier U35A, the first terminal of resistor R65A and the first terminal of resistor R66A. The second terminal of resistor R65A is connected to the non-inverting terminal of instrumentation amplifier U36A, and the second terminal of resistor R66A is connected to the first terminal of capacitor C65A and the inverting terminal of instrumentation amplifier U36A. The second terminal of capacitor C65A is connected to circuit ground.

The reference terminal of instrumentation amplifier U36A is connected to ground. The gain of instrumentation amplifier U36A is set at 5 by leaving the Rg terminals unconnected for the AD627 (Analog Devices, Norwood, Mass., USA). The output terminal of instrumentation amplifier U36A is connected to the non-inverting input terminal of instrumentation amplifier U37A. The non-inverting input terminal of instrumentation amplifier U37A is connected to the output terminal of instrumentation amplifier U36n.

Continuing in measurement channel n, for the “BCG” compensation channel, the second terminal of resistor R60n is connected to the first terminal of capacitor C61n and the first terminal of resistor R61n. The second terminal of capacitor C61n is connected to the inverting input of operational amplifier U33n. The second terminal of resistor R61n is connected to the first terminal of capacitor C60n and the non-inverting input of operational amplifier U33n. The second terminal of capacitor C60n is connected to circuit ground. The output terminal of operational amplifier U33n is connected to the inverting input of operational amplifier U33n and the first terminal of resistor R62n. The second terminal of resistor R62n is connected to the first terminal of capacitor C63n and the first terminal of resistor R63n. The second terminal of capacitor C63n is connected to the inverting input of operational amplifier U34n. The second terminal of resistor R63n is connected to the first terminal of capacitor C62n and the non-inverting input of operational amplifier U34n. The second terminal of capacitor C62n is connected to circuit ground.

The output terminal of operational amplifier U34n is connected to the inverting input of operational amplifier U34n and the first terminal of resistor R64n. The second terminal of resistor R64n is connected to the first terminal of capacitor C65n and the first terminal of resistor R65n. The second terminal of capacitor C65n is connected to the inverting input of operational amplifier U35n. The second terminal of resistor R65n is connected to the first terminal of capacitor C64n and the non-inverting input of operational amplifier U35n. The second terminal of capacitor C64n is connected to circuit ground.

The output terminal of operational amplifier U35n is connected to the inverting input of operational amplifier U35n, the first terminal of resistor R65n and the first terminal of resistor R66n. The second terminal of resistor R65n is connected to the non-inverting terminal of instrumentation amplifier U36n, and the second terminal of resistor R66n is connected to the first terminal of capacitor C65n and the inverting terminal of instrumentation amplifier U36n. The second terminal of capacitor C65n is connected to circuit ground. The reference terminal of instrumentation amplifier U36n is connected to ground. The gain of instrumentation amplifier U36n is set at 5 by leaving the Rg terminals unconnected for the AD627 (Analog Devices, Norwood, Mass., USA). The output terminal of instrumentation amplifier U36n is connected to the non-inverting input terminals of instrumentation amplifiers U37 in the measurement channels.

For the second compensation channel derived from channel n (powerline), the first terminal of resistor R70 is connected to terminal 12 of filter module U37. The second terminal of resistor R70 is connected to terminal 13 of U37. The first terminal of resistor R71 is connected to terminal 13 of U37. The second terminal of resistor R71 is connected to terminal 8 of U37. The first terminal of resistor R74 is connected to terminal 3 of U37. The second terminal of resistor R74 is connected to circuit ground. The first terminal of resistor R73 is connected to terminal 7 of U37. The second terminal of resistor R73 is connected to terminal 14 of U37.

Terminal 7 of U37 is connected to the first terminal of resistor R75 and the first terminal of resistor R76. The second terminal of resistor R75 is connected to the first terminal of capacitor C70 and the non-inverting terminal of operational amplifier U38. The second terminal of resistor R76 is connected to the first terminal of resistor R77 and the inverting terminal of operational amplifier U38. The second terminal of resistor R77 is connected to the first terminal of resistor R80, the output of U38 and the first terminal of capacitor C71. The second terminal of capacitor C71 is connected to the first terminal of variable resistor R78. The second terminal of variable resistor R78 is connected to the wiper terminal of variable resistor R78 and the first terminal of variable resistor R79. The second terminal and wiper terminal of variable resistor R79 is connected to circuit ground. The second terminal of resistor R80 is connected to the non-inverting input of operational amplifier U39 and the first terminal of resistor R81. The second terminal of resistor R81 is connected to the output of U39 and the first terminal of variable resistor R82.

The wiper terminal of variable resistor resistor R82 is connected to the non-inverting terminal of operational amplifier U40. The second terminal of variable resistor R82 is connected to the first terminal of resistor R83. The second terminal of resistor R83 is connected to circuit ground. The non-inverting input terminal of operational amplifier U40 is connected to the output terminal of U40. For the powerline compensation signal to be used in measurement channel 1, the first terminals of resistor R90A and the first terminal of resistors R91A are connected to the output terminal of U40. The second terminal of resistor R90A is connected to the first terminal of resistor R92A and the second terminal of variable resistor R91A. The second terminal of resistor R92A is connected to circuit ground. The wiper terminal of variable resistor R91A is connected to the non-inverting input terminal of operational amplifier U41A. The inverting input terminal of U41A is connected to the output terminal of U41A and the reference terminal of instrumentation amplifier U30A.

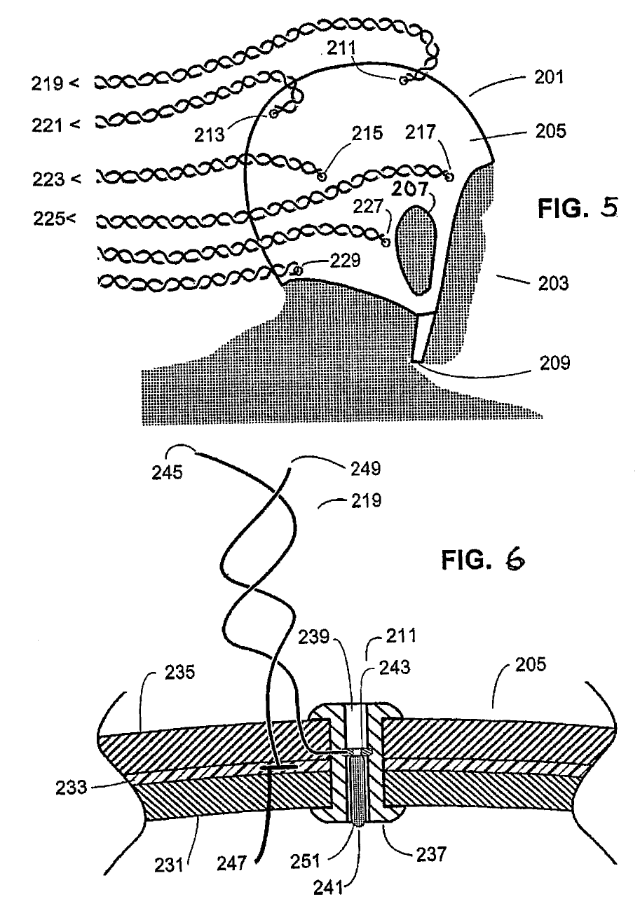

In FIG. 5, an electrode support cap 201 in accordance with the present invention is shown in place on the head 203 of a subject. It comprises a flexible head covering piece 205 provided with holes such as 207 etc for the ears. The cap is retained on the head by means of a chin strap 209. Four measurement signal/reference node pairs are provided spatially separated over the surface of the cap, denoted by reference numerals 211, 213, 215 and 217. Each of these pairs is connected to external circuitry by means of twisted wire pairs 219, 221, 223, 225.

A separate compensation electrode with associated reference electrode with its own twisted wire pair for external connection is denoted by numeral 227. This is located just behind the right ear.

At the base of the neck region of the headpiece 205, is arranged a ground electrode/reference electrode pair 229, again with a twisted wire pair connection to remote circuitry.

A cross-section through one measurement electrode/reference node pair 211 is shown in FIG. 6.

As can be seen in this cross-sectional view, the flexible cap headpiece 205 comprises an insulating nylon stretch fabric base layer 231, on top of which is situated a silver coated nylon reference mesh 223. Above this, is situated an upper stretch fabric netting 235.

This three layer structure 231, 233, 235 is provided with a hole bridged by a cylindrical grommet 237 of suitable insulating material. A central bore 239 runs axially through the centre of the grommet. The lower part of this bore is filled with a conductive gel 241, on top of and in electrical contact therewith, being a measurement electrode metal insert 243 which exits the side wall of the grommet, upwardly through the stretch fabric netting layer 235 to be connected to measurement signal wire 245 forming one half of the twisted wire pair 219.

Immediately adjacent the grommet 237 is located a reference electrode (node) connection 247, embedded in the conductive silver coated reference mesh layer 233, which is in electrical contact with wire 249 which exits through the upper stretch fabric netting 235, twisted with the measurement signal wire 245 to form the other half of twisted wire pair 219.

In use, the lower part 251 of the conductive gel 241 is in contact with the scalp of the subject.

In the light of the described embodiments, modifications of those embodiments, as well as other embodiments, all within the scope of the appended claims as interpreted in the light of the specification as a whole and with the knowledge of a person skilled in the art, will now become apparent.

1. An electronic circuit for reducing interference in a measurement signal or signals, wherein the interference comprises a plurality of interference components, the electronic circuit comprising:
(a) at least one primary signal processing unit, the or each primary signal processing unit having a respective measurement signal input for receiving a respective one of said measurement signal or signals and the or each primary signal processing unit comprising a plurality of interference reduction modules;
(b) a respective compensation signal component input for each interference reduction module;
(c) a compensation signal processing unit having at least one compensation signal input and comprising means for deriving from at least one compensation signal, a plurality of compensation signal components each of which is related to a respective one or more of the interference components; and
(d) the compensation signal processing unit also having a respective compensation signal component output for each compensation signal component, each said output being respectively connected to one of the compensation signal component inputs.

2. The electronic circuit of claim 1, wherein in each primary signal processing unit, the interference reduction modules are arranged in series.

3. The electronic circuit of claim 1, wherein in each primary signal processing unit, respective interference reduction modules are provided for reduction of at least two of rf interference, magnetic field switching interference, mains power interference, electrode and/or lead movement, eyeblink artifact interference and ballistocardiogram interference, respectively.

4. The electronic circuit of claim 1, wherein a respective measurement signal electrode is connected to the or each measurement signal input of the at least one primary signal processing unit via a measurement signal line and is in direct electrical contact with a subject and for each measurement signal line or group of signal lines, a corresponding reference signal electrode is connected via a reference signal line to a respective reference signal input of the at least one primary signal processing unit.

5. The electronic circuit of claim 4, wherein the or each primary signal unit further comprises subtraction means for subtracting at least part of a signal on the respective reference signal line from the signal on the corresponding respective measurement signal line or lines.

6. The electronic circuit of claim 4, wherein the compensation signal input is connected via a compensation signal line to a compensation signal electrode in direct electrical connection with a subject and a circuit ground connection is connected via a ground line to a ground electrode, respective reference signal lines being arranged in close proximity with the compensation signal line and ground line along respective substantial parts of the length thereof, the reference signal lines being connected to respective reference electrodes.

7. The electronic circuit of claim 4, wherein a respective ground line is arranged in associated close proximity with the or each signal line along a substantial part of the length thereof, and a further ground line is arranged in associated close proximity with the or each reference signal line along a substantial part of the length thereof, each of the ground lines being connected to one or more ground electrodes in direct or indirect electrical contact with the subject.

8. The electronic circuit of claim 4, wherein a respective signal ground line is associated in close proximity with the or each measurement signal line/reference line pair along a substantial part of the length thereof, each of the ground lines being connected to one or more ground electrodes in direct or indirect electrical contact with the subject.

9. The electronic circuit of claim 8, wherein the circuit ground connections of the ground lines associated with the signal lines and associated grounds are electrically isolated from the circuit ground connections of the reference lines.

10. The electronic circuit of claim 6, wherein each measurement signal line is twisted together with its respective reference line and the ground signal line and compensation signal line are twisted together with their respective reference lines.

11. The electronic circuit of claim 10 where all of the measurement signal line/reference line pairs, the compensation signal line reference line pair and the ground line/reference line pair are twisted together.

12. (canceled)

13. (canceled)

14. (canceled)

15. The electronic apparatus of claim 6, wherein the or each measurement signal line/reference signal line pair is shielded.

16. The electronic circuit of claim 4, wherein for at least some signal line/reference line pairs, at least one additional reference line is provided, connected to the same or a respective further reference electrode.

17. A combined measurement apparatus comprising an MRI, TMS or MEG unit and an EPM system which comprises an electronic circuit for reducing interference in a measurement signal or signals, wherein the interference comprises a plurality of interference components, the electronic circuit comprising:
(a) at least one primary signal processing unit, the or each primary signal processing unit having a respective measurement signal input for receiving a respective one of said measurement signal or signals and the or each primary signal processing unit comprising a plurality of interference reduction modules;
(b) a respective compensation signal component input for each interference reduction module;
(c) a compensation signal processing unit having at least one compensation signal input and comprising means for deriving from at least one compensation signal, a plurality of compensation signal components each of which is related to a respective one or more of the interference components; and
(d) the compensation signal processing unit also having a respective compensation signal component output for each compensation signal component, each said output being respectively connected to one of the compensation signal component inputs.

18. The combined apparatus of claim 17, wherein the MRI unit is adapted for fMRI and wherein the EPM system is selected from systems for effecting one or more of EEG, ECG, EMG, EOG, ERG and GSR.

19. (canceled)

20. The electronic circuit of claim 1 wherein a plurality of said measurement signal inputs are connected to receive respective measurement signals from an array of measurement signal electrodes supported on an electrode support apparatus so as to be presented for contacting the skin of a subject, first connection means being provided for independent electrical connection to each of said measurement signal electrodes, the support apparatus further comprising an electrically conductive mesh having one or more of reference nodes and second connection means for independent electrical connection to the or each of said reference nodes.

21. (canceled)

22. The electronic circuit of claim 21, wherein the number of said reference nodes is substantially the same as the number of said measurement signal electrodes and wherein each measurement signal electrode or group of signal electrodes has a corresponding respective reference node in close physical proximity thereto.

23. (canceled)

24. The electronic circuit of claim 21, wherein said electrode support further supports one or more ground electrodes presented for contacting the skin of a subject, the apparatus further comprising third connection means for independent electrical connection to each of said ground electrode or electrodes.

25. The electronic circuit of claim 21, wherein the electrode support supports a single ground electrode and wherein the electrode support supports at least one compensation signal electrode.

26. (canceled)

27. The electronic circuit of claim 21, wherein the electrode support supports a single ground electrode and at least one compensation signal electrode and wherein a respective reference node with its own independent electrical connection is provided for the ground electrode and the compensation signal electrode.

28. The electronic circuit of claim 21, wherein said mesh comprises a continuous laminar member comprising said reference nodes.

29. The electronic circuit of claim 21, wherein said mesh comprises a matrix of discrete members respectively comprising said reference nodes.

30. The electronic circuit of claim 21, wherein said electrode support is in the form of a flexible cap.

31. The electronic circuit of claim 21, comprising a rigid cap, the conductive mesh being flexible.

32. A method of reducing interference in a measurement signal or signals, wherein the interference comprises a plurality of interference components, the method comprising:
(a) inputting the at least one measurement signal to a respective primary signal processing unit, the or each primary signal processing unit comprising a plurality of interference reduction modules each having a compensation signal component input;
(b) inputting at least one compensation signal to a compensation signal processing unit wherein a plurality of compensation signal components are derived from the at least one compensation signal, each compensation signal component being related to a respective one or more of the interference components; and
(c) inputting the compensation signal components to respective compensation signal component inputs of the at least one primary signal processing unit.
